(19) United States
(12) Patent Application Publication
(10) Pub. No.: US 2011/0189221 A1
(43) Pub. Date: Aug. 4, 2011

(54) NOVEL ANTIVIRAL COMPOUNDS FROM MARINE EXTRACTS

(76) Inventors: Alberto van Olphen, Tampa, FL (US); Bill J. Baker, Tampa, FL (US); Dennis E. Kyle, Lithia, FL (US); Cynthia Bucher, Temple Terrace, FL (US); Alan Maschek, Tampa, FL (US); James B. McClintock, JR., Birmingham, AL (US); Charles D. Amsler, Pelham, AL (US)

(21) Appl. No.: 13/007,380

(22) Filed: Jan. 14, 2011

Related U.S. Application Data
(63) Continuation-in-part of application No. 12/610,657, filed on Nov. 2, 2009.
(60) Provisional application No. 61/110,310, filed on Oct. 31, 2008.

(51) Int. Cl. A61K 36/02; A61P 31/16; C07K 7/08; C07K 7/06; C07K 14/405; C12Q 1/70

(52) U.S. Cl. 424/195.17; 530/326; 530/327; 530/328; 530/324; 530/329; 435/5

(57) ABSTRACT

The subject invention pertains to novel biologically active extracts from marine algae and to biologically active fractions and components of these extracts. These extracts have been shown to possess antiviral properties. Pharmaceutical compositions comprising these extracts, or comprising biologically active fractions or components of these extracts, could be used in the treatment of viral diseases including influenza.

### CROSS-REFERENCE TO RELATED APPLICATIONS

This application is a continuation-in-part of co-pending application U.S. Ser. No. 12/610,657, filed Nov. 2, 2009, which claims the benefit of U.S. Provisional Application Ser. No. 61/110,310, filed Oct. 31, 2008, which are incorporated herein by reference in their entirety.

### GOVERNMENT SUPPORT

The subject invention was made with government support under Grant No. W911 SR-06-C-0020 awarded by the U.S. Army, and Grant No. OPP-0442857 awarded by the National Science Foundation, Office of Polar Programs. The government has certain rights in the invention.

### FIELD OF THE INVENTION

This invention relates to marine extracts, and components of such extracts, which have useful therapeutic properties. More particularly, the invention concerns novel marine extracts having anti-viral activity, pharmaceutical compositions comprising such extracts or components of such extracts, derivatives of the extracts or the components, and methods of use for therapeutic purposes.

### BACKGROUND OF THE INVENTION

Viral diseases afflict man, plants, insects, and animals. The prevention and control of viral diseases has important health and economic implications. Viral diseases contribute to afflictions in humans including the common cold, herpes, acquired immune deficiency syndrome (AIDS), and cancer. Also important is the control of viral diseases in animals for economic and other reasons, e.g., the ability of such animals to become virus reservoirs or carriers which facilitate the spreading of viral diseases to humans. Viral plant diseases have been known to have a disruptive effect on the cultivation of fruit trees, tobacco, and various vegetables. Insect viral diseases are also of interest, in part because of the insects' ability to transfer viral diseases to humans.

The prevention and control of viral diseases is thus of prime importance to man, and considerable research has been devoted to antiviral measures. Certain methods and chemical compositions have been developed which aid in inhibiting, controlling, or destroying viruses, but additional methods and antiviral compositions are needed.

Worldwide afflictions due to the influenza virus illustrate the need for new and effective therapeutics against viruses. The fear of a pandemic outbreak, the seasonal epidemics, and the emergence of drug-resistant strains underscore this urgent need. The economic impact caused by influenza due to decreased productivity and increased health care utilization is estimated to be in the billions of dollars. The World Health Organization (WHO) has estimated that 3 to 5 million people are infected with influenza each year, and as many as 500,000 people die from the complications of these infections. The influenza outbreak of 1918-19, the deadliest on record, killed about 40 million people worldwide, including about 650,000 in the United States. Currently, scientists fear that the new avian influenza H5N1 could mutate into a strain that easily transmits from person to person, sparking a human influenza pandemic resulting in devastating human and economic consequences. According to the WHO, since the initial outbreak in South East Asia in 1997 until Nov. 13, 2006, the H5N1 virus has thus far spread to at least ten countries and caused the death of 153 people and the mandatory slaughtering of millions of birds.

In searching for new biologically active compounds, it has been found that some natural products and organisms are potential sources for chemical molecules having useful biological activity of great diversity. For example, the diterpene commonly known as paclitaxel, isolated from several species of yew trees, is a mitotic spindle poison that stabilizes microtubules and inhibits their depolymerization to free tubulin (Fuchs, D. A., R. K. Johnson [1978] Cancer Treat. Rep. 62:1219-1222; Schiff, P. B., J. Fant, S. B. Horwitz [1979] Nature (London) 22:665-667). Paclitaxel is also known to have antitumor activity and has undergone a number of clinical trials which have shown it to be effective in the treatment of a wide range of cancers (Rowinski, E. K. R. C. Donehower [1995]N. Engl. J. Med. 332:1004-1014). See also, e.g., U.S. Pat. Nos. 5,157,049; 4,960,790; and 4,206,221.

Marine sponges have also proven to be a source of biologically active chemical molecules. A number of publications disclose organic compounds derived from marine sponges including Scheuer, P. J. (ed.) Marine Natural Products, Chemical and Biological Perspectives, Academic Press, New York, 1978-1983, Vol. I-V; Uemura, D., K. Takahashi, T. Yamamoto, C. Katayama, J. Tanaka, Y. Okumura, Y. Hirata (1985) J. Am. Chem. Soc. 107:4796-4798; Minale, L. et al. (1976) Fortschr. Chem. org. Naturst. 33:1-72 Faulkner, D. J., Nat. Prod. Reports 1984, 1, 251-551; ibid. 1987, 4, 539; ibid 1990, 7, 269; ibid 1993, 10, 497; ibid 1994, 11, 355; ibid 1995, 12, 22; ibid 1998, 15:113-58; ibid 2000 17:1-6; ibid 2000 17: 7-55; ibid 2001, 18: 1-49; 2002, 19: 1-48.; Gunasekera, S. P., M. Gunasekera, R. E. Longley and G. K. Schulte (1990) J. Org. Chem., 55:4912-4915.; Horton, P. A., F. E. Koehn, R. E. Longley, and O. J. McConnell, (1994) J. Am. Chem. Soc. 116: 6015-6016.

Likewise, other marine organisms, including algae, have been reported as sources of biologically active compounds. Exemplary publications include Park, H. J., Kurokawa, M., Shiraki, K., Nakamura, N., Choi, J. S., and Hattori, M. (2005), Antiviral activity of the marine alga Symphyocladia latiuscula against Herpes simplex virus (HSV-1) in vitro and its therapeutic efficacy against HSV-1 infection in mice, Biol. Pharm. Bull. 28, 2258-2262; Serkedjieva, J. (2004), Antiviral activity of the red marine alga Ceramium rubrum. Phytother. Res 18, 480-483; Toranzo, A. E., Barja, J. L., and Hetrick, F. M. (1982), Antiviral activity of antibiotic-producing marine bacteria, Can. J Microbiol. 28, 231-238; Wright, A. D., Konig, G. M., Angerhofer, C. K., Greenidge, P., Linden, A., and Desqueyroux-Faundez, R. (1996), Antimalarial activity: the search for marine-derived natural products with selective antimalarial activity, J. Nat. Prod. 59, 710-716; and Pujol, C. A., Scolaro, L. A., Ciancia, M., Matulewicz, M. C., Cerezo, A. S., Damonte, E. B. (2006), Antiviral activity of a carrageenan from Gigartina skottsbergii against intraperitoneal murine Herpes simplex virus infection, Planta Medica 72, 121-125.

### BRIEF SUMMARY

The subject invention pertains to novel biologically active extracts from marine algae and to biologically active fractions and components of these extracts. These extracts have been shown to possess anti-viral properties. In one embodiment, the subject invention provides a Gigartina extract and anti-viral compounds (e.g., proteins) contained in the Gigartina extract. In a specific embodiment, the Gigartina extract and anti-viral compounds (e.g., proteins) contained in the Gigartina extract are prepared using rhodophyte Gigartina skottsbergii Setchell & Gardner 1936 (Phylum: Rhodophyta, Class: Florideophyceae, Sub Class: Rhodymeniophycidae, Order: Gigartinales, Family: Gigartinaceae).

The subject invention further provides pharmaceutical compositions comprising these extracts, or comprising biologically active fractions or components of these extracts, which can be used in the prevention and/or treatment of viral diseases including influenza.

The subject invention provides biologically active marine extracts, and biologically active fractions or components thereof, that may be obtained according to any of the following procedures:

(i) A 2001 collection (PSC01-12) of frozen algae (2.2 kg) was extracted with CH₂Cl₂/MeOH (1:1, 1 L×3). The combined extract was concentrated to a dark green crude (3.6 g). The residue was subjected to Si gel column chromatography with a hexanes/EtOAc/MeOH gradient solvent system to give 13 fractions. Fraction 9 (PSC01-12-6-I, 309.3 mg) eluted with approximately 50% EtOAc/MeOH then was fractionated by RP HPLC(YMC-PAK ODS-AQ) with a gradient of 50% aqueous MeOH to 100% MeOH to yield 9 fractions. A 2007 collection (PSC07-52) of fresh algae (12.1 kg) was extracted with MeOH (4 L×3). The combined extract was concentrated (344.4 g), and the residue was partitioned between CH₂Cl₂ and H₂O, Subsequently, the CH₂Cl₂ layer was concentrated in vacuo to give a dark green crude (PSC07-52-A, 20.2 g). The residue was subjected to Si gel column chromatography with a hexanes/EtOAc/MeOH gradient solvent system to give 12 fractions. Fraction 9 (PSC07-52-A-I, 2.9 g) eluted with approximately 50% EtOAc/MeOH then fractionated by RP HPLC (YMC-PAK ODS-AQ) with a gradient of 50% aqueous MeOH to 100% MeOH to yield 8 fractions. A 2008 collection (PSC08-08-A) of fresh algae was extracted with MeOH (4 L×3). The combined extract was concentrated and the residue was partitioned between CH₂Cl₂ and H₂O, Subsequently, the CH₂Cl₂ layer was concentrated in vacuo to give a dark green crude (PSC08-8-A-A, 6.5 g). The residue was subjected to Si gel column chromatography with a hexanes/EtOAc/MeOH gradient solvent system to give 12 fractions. Fractions 9 and 10 (PSC08-8-A-A-9, 414 mg, and PSC08-8-A-A-10, 178 mg) were identified as active fractions.
(ii) A process comprising seven steps for obtaining, purifying, or concentrating the active compound was developed (FIG. 6). Whole algae thalli were used for the preparation of protein extracts using the commercial kit P-PER (Pierce). 2) The aqueous phase of this extraction demonstrated antiviral activity. 3) Subsequently it was further fractionated using membrane filtration as follows: The aqueous phase was sequentially 4) filtered through 30,000 Dalton Amicon membrane. What remains unfiltered is named retentate 1 and the material that passes through the filter is called filtrate 1. 5). Filtrate 1 was passed through a 10,000 Dalton Amicon filter resulting in retentate 2 and filtrate 2. 6). Filtrate 2 was passed through a 3000 Dalton Amicon filter producing retentate 3 and filtrate 3. 7). Retentate 1, 2 and 3 and filtrate 1 and 2 presented antiviral activity indicating that the active compound has a molecular size larger than 3,000 Daltons. One aspect of the current invention concerns the composition, and component compounds thereof, as prepared in (i) above. Advantageously, this composition (through the action of its component compounds) can inhibit, control, or destroy viruses, including influenza virus.

The compositions of the invention can be administered as a treatment for existing viral infections, or as prophylaxis (for preventing or delaying the onset of viral infections), in human and non-human mammals; alternatively, they may be used in vitro to inhibit viruses. In a specific embodiment, the compositions and methods of the subject invention can be used in the treatment of an animal afflicted with a viral infection including, for example, inhibiting the production of viral progeny in a mammalian host. More particularly, the subject compounds can be used in a human for inhibiting, controlling, or destroying viruses, including for example influenza virus. The probable mechanisms for achieving antiviral activity exhibited by the subject compounds would lead a person of ordinary skill in the art to recognize the applicability of the subject compounds, compositions, and methods to additional types of viruses that are described herein, or are otherwise well-known in the art, or may become known in the art.

In specific embodiments, the subject invention provides new compounds, as exemplified by the composition prepared in (i) above. Such compounds have not been isolated previously from a natural source nor have they been previously synthesized. One embodiment of the subject invention provides a mixture of any of the component compounds obtainable according to (i) through protein extraction and fractionation (i and ii) above, wherein the mixture exhibits the desired antiviral activity.

In one embodiment, the subject invention provides bioactive compounds (e.g., proteins) contained in the Gigartina extract. In a specific embodiment, the proteins of the subject invention comprise one or more amino acid sequences selected from SEQ ID NO:1 to SEQ ID NO:18. Advantageously, the bioactive compounds (e.g., proteins) of the subject invention have anti-viral effects. For instance, the bioactive compounds can inhibit, control, or destroy viruses, including influenza. Also provided are compositions (including Gigartina extract) that comprise proteins of the subject invention.

In accordance with the subject invention, methods for inhibiting, controlling, or destroying viruses in a host include contacting virally-infected cells with an effective amount of the new pharmaceutical compositions of the invention. The viruses inhibited by the invention are those which are susceptible to the subject compounds described herein or compositions comprising those compounds.

Additional aspects of the invention include the provision of methods for producing the new compounds and compositions.

Other objects and further scope of applicability of the present invention will become apparent from the detailed descriptions given herein; it should be understood, however, that the detailed descriptions, while indicating preferred embodiments of the invention, are given by way of illustration only, since various changes and modifications within the spirit and scope of the invention will become apparent from such descriptions.

In one embodiment of the present invention, a composition can comprise an extract of a Gigartina species. In a specific embodiment, the composition of the present invention comprises an extract of Gigartina skottsbergii, wherein said extract comprises a component having a molecular weight greater than 3,000 Daltons, and wherein said extract exhibits one or more anti-viral properties.

An embodiment of the present invention is an extract that inhibits viral growth.

In another embodiment, said extract inhibits influenza replication.

In yet another embodiment, the composition comprises a protein.

In an embodiment of the present invention, a composition isolated from Gigartina skottsbergii, wherein said compound has a molecular weight greater than 3,000 Daltons, and wherein said compound exhibits one or more anti-viral properties.

An embodiment of the present invention is a composition that inhibits viral growth.

In another embodiment, said composition inhibits influenza replication.

In yet another embodiment, the composition comprises a protein.

In an embodiment of the present invention, a method for treating or preventing a viral infection in a mammalian subject can include administering to the subject in need thereof a composition comprising an extract of Gigartina skottsbergii, wherein said extract comprises a component having a molecular weight greater than 3,000 Daltons, and wherein said extract exhibits one or more anti-viral properties; or a composition isolated from Gigartina skottsbergii, wherein said compound has a molecular weight greater than 3,000 Daltons, and wherein said compound exhibits one or more anti-viral properties.

An embodiment of the present invention, wherein the viral infection is influenza.

In an embodiment of the present invention, a method is provided for obtaining an extract having antiviral activity against one or more viruses of interest, comprising
(a) providing one or more biological extracts;
(b) carrying out a primary screening on each extract, comprising:
   i) evaluating cytopathic effects (CPE) of a virus on host cells in the presence of each extract in vitro relative to a control;
   ii) carrying out a quantitative cell viability assay and, optionally, crystal violet staining;
(c) carrying out a secondary screening on each extract identified as exhibiting protection against CPE in the primary screening, comprising
   i) carrying out a dose response assay on each extract;
   ii) carrying out a plaque reduction assay on each extract; and
   iii) carrying out one-step virus progeny production;
   iv) carrying out a selectivity evaluation comprising cytotoxicity assay.

An embodiment of the current invention wherein the composition comprises repeating (b) and (c) one or more times on each extract determined to have antiviral activity, using a different virus of interest.

In another embodiment, the composition comprises repeating one or more times the primary screening of (b) on each extract identified as exhibiting protection against CPE in the primary screening.

In yet another embodiment, the composition comprises an extract identified as exhibiting protection against CPE in the primary screening if the extract provides at least 50% protection at 100 μg/mL.

In another embodiment, the primary screening of (a), the secondary screening of (b), or both (a) and (b) carried out on a plurality of serial dilutions of the extract.

In yet another embodiment, the extract is a bacterial extract.

In another embodiment, the extract is an algae extract.

In yet another embodiment, the extract is a marine extract.

In another embodiment, the virus of interest is influenza virus.

In yet another embodiment, the virus of interest is influenza A (e.g., H1N1 & H3N2) and influenza B viruses.

### BRIEF DESCRIPTION OF THE FIGURES

FIG. 1 shows a crystal violet stained plate and optical reading of the corresponding cell viability assay results for a sample plate.

FIG. 2 illustrates a microtiter plate showing assay results.

FIG. 3 shows plates after performance of a plaque reduction assay.

FIG. 4 illustrates the screening pathway and procedures.

FIG. 5 shows a 96-well plate representing the primary screen for marine extracts.

FIG. 6A shows a schematic of a processes for the isolation and purification of the active fraction of Gigartina spp. displaying anti-viral activity. FIG. 6B shows tryptic digestion of proteins contained in the Gigartina extract.

FIG. 7 shows the extract fractionation scheme using a hexane/EtOAc/MeOH gradient solvent system.

FIGS. 8A-8E show that, after tryptic digestion of proteins contained in the Gigartina extract, the resulting peptides comprise amino acid sequences (shown as highlighted) that are also part of an ubiquitin-like protein, a griffithsin-like protein, an alkyl hydroperoxide reductase subunit C-like protein, a phycoerythrin beta chain-like protein, and/or a beta-N-acetylhexosaminidase-like protein.

FIGS. 9A-B show expression and purification of C. elegans homolog of the ubiquitin-like protein.

FIGS. 10A-D show mass spectra of the Griffithsin-like protein contained in the Gigartina extract. (A) shows ESI-MS charge state distribution of the 14.49 k protein. (B) shows expanded m/z region, showing isotope distribution for +16 charge state of the Griffithsin-like protein contained in the Gigartina extract. (C) shows mass spectrum of the Griffithsin-like protein after deconvolution. (D) shows MS/MS of fragment ion m/z 856.9, which displays a fragmentation pattern for above 18 amino acid sequence. This Griffithsin-like protein fraction presents anti-influenza activity.

### DETAILED DESCRIPTION OF THE SEQUENCES

SEQ ID NO:1 is a partial amino acid sequence of a protein contained in the Gigartina extract.

SEQ ID NO:2 is a partial amino acid sequence of a protein contained in the Gigartina extract.

SEQ ID NO:3 is a partial amino acid sequence of a protein contained in the Gigartina extract.

SEQ ID NO:4 is a partial amino acid sequence of a protein contained in the Gigartina extract.

SEQ ID NO:5 is a partial amino acid sequence of a protein contained in the Gigartina extract.

SEQ ID NO:6 is a partial amino acid sequence of a protein contained in the Gigartina extract.

SEQ ID NO:7 is a partial amino acid sequence of a protein contained in the Gigartina extract.

SEQ ID NO:8 is a partial amino acid sequence of a protein contained in the Gigartina extract.

SEQ ID NO:9 is a partial amino acid sequence of a protein contained in the Gigartina extract.

SEQ ID NO:10 is a partial amino acid sequence of a protein contained in the Gigartina extract.

SEQ ID NO:11 is a partial amino acid sequence of a protein contained in the Gigartina extract.

SEQ ID NO:12 is a partial amino acid sequence of a protein contained in the Gigartina extract.

SEQ ID NO:13 is a partial amino acid sequence of a protein contained in the Gigartina extract.

SEQ ID NO:14 is a partial amino acid sequence of a protein contained in the Gigartina extract.

SEQ ID NO:15 is a partial amino acid sequence of a protein contained in the Gigartina extract.

SEQ ID NO:16 is a partial amino acid sequence of a protein contained in the Gigartina extract.

SEQ ID NO:17 is a partial amino acid sequence of a protein contained in the Gigartina extract.

SEQ ID NO:18 is a partial amino acid sequence of a protein contained in the Gigartina extract.

### DETAILED DISCLOSURE OF THE INVENTION

The subject invention pertains to novel extracts obtained from marine algae, and particulary to novel extracts obtained from a Gigartina species, such as for example, Gigartina skottsbergii. These extracts have been shown to possess potent anti-viral properties, especially against influenza virus. The subject invention pertains to the extracts themselves, components of the extracts, and pharmaceutical compositions containing the extracts or components. Also disclosed and claimed are methods for producing the extracts, components, and compositions. Various derivatives of the extracts, components, or compositions can be produced by procedures known in the art.

Gigartina species useful according to the subject invention include, but are not limited to, Gigartina skottsbergii, Gigartina intermedia, Gigartina exasparata, Gigartina acicularis, Gigartina pistillata, Gigartina radula, Gigartina stellata, and Gigartina acicularis.

In a specific embodiment, the Gigartina extract and anti-viral compounds (e.g., proteins) contained in the Gigartina extract are prepared using rhodophyte Gigartina skottsbergii Setchell & Gardner 1936 (Phylum: Rhodophyta, Class: Florideophyceae, Sub Class: Rhodymeniophycidae, Order: Gigartinales, Family: Gigartinaceae).

In a specific embodiment, the extracts, components, or compositions of the subject invention comprise proteins having a molecular size larger than 3,000 Daltons. In a further specific embodiment, proteins contained in the Gigartina extract have anti-viral effects, and comprise one or more amino acid sequences selected from SEQ ID NO:1 to SEQ ID NO:18, or fragments thereof.

In addition, the subject invention provides methods for prevention and/or treatment of viral infection including, but not limited to, influenza. In one embodiment, the method comprises administering, to a subject in need of such treatment, an effective amount of the compounds and/or compositions of the subject invention.

The subject invention provides novel compositions of biologically active compounds that are useful for inhibiting, controlling, or destroying viruses. In a preferred embodiment, these compounds can be used for inhibiting, controlling, or destroying influenza virus. Plants, animals, microbes, or any other living organism may be treated.

More specifically, the novel compounds, compositions, and methods of use can advantageously be used to inhibit, control, or destroy influenza virus and other viruses in a mammalian host. More particularly, the subject compounds can be used for inhibiting, controlling, or destroying virus that is present in a human, including influenza virus. The compounds also have utility in the treatment of viruses that are resistant to known antiviral therapies.

Additional viruses that can be treated according to the invention are those that have been classified by the International Committee on Taxonomy of Viruses (ICTV). Those of skill in the art will recognize that the ICTV periodically publishes information on viruses in printed publications and through the internet. For example, “Virus Taxonomy: VIIIth Report of the International Committee on Taxonomy of Viruses”, 2005, C. M. Fauquet, M. A. Mayo, J. Maniloff, U. Desselberger, and L. A. Ball (Eds), Elsevier Academic Press, is such a publication and is incorporated herein by reference in its entirety. It is envisioned that the invention may be employed to treat each of various functional and structural sub-classes of viruses as identified by the ICTV, and each individual virus. In a preferred embodiment, the invention may be employed to treat viruses belonging to each of the classes and subclasses to which an influenza virus belongs, or to which a specific influenza virus tested in the examples herein belongs.

The subject invention provides biologically active marine extracts, and biologically active fractions or components thereof, that may be obtained according to any of the following procedures:

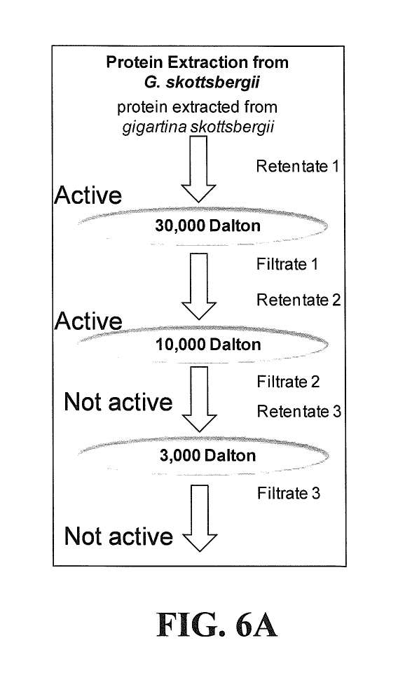

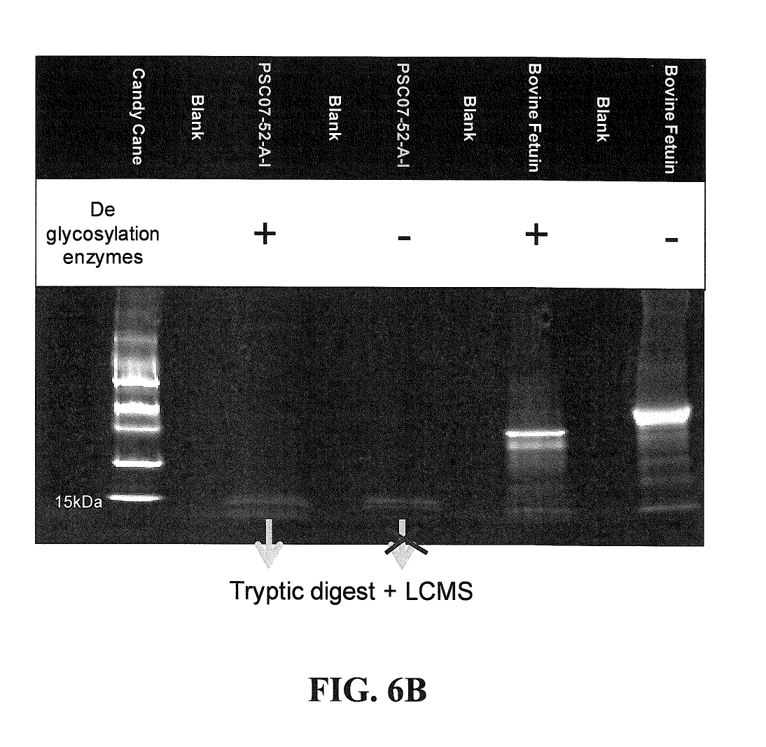

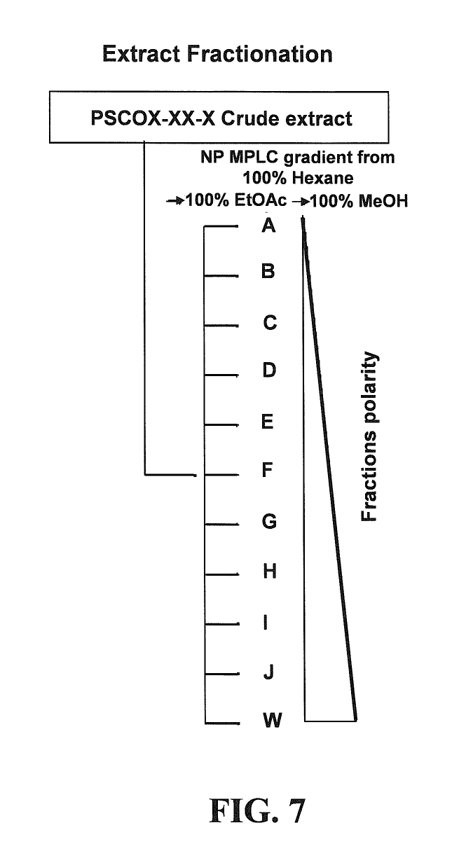

(i) A 2001 collection (PSC01-12) of frozen algae (2.2 kg) was extracted with CH₂Cl₂/MeOH (1:1, 1 L×3). The combined extract was concentrated to a dark green crude (3.6 g). The residue was subjected to Si gel column chromatography with a hexanes/EtOAc/MeOH gradient solvent system to give 13 fractions. Fraction 9 (PSC01-12-6-I, 309.3 mg) eluted with approximately 50% EtOAc/MeOH then was fractionated by RP HPLC (YMC-PAK ODS-AQ) with a gradient of 50% aqueous MeOH to 100% MeOH to yield 9 fractions. A 2007 collection (PSC07-52) of fresh algae (12.1 kg) was extracted with MeOH (4 L×3). The combined extract was concentrated (344.4 g), and the residue was partitioned between CH₂Cl₂ and H₂O, Subsequently, the CH₂Cl₂ layer was concentrated in vacuo to give a dark green crude (PSC07-52-A, 20.2 g). The residue was subjected to Si gel column chromatography with a hexanes/EtOAc/MeOH gradient solvent system to give 12 fractions. Fraction 9 (PSC07-52-A4, 2.9 g) eluted with approximately 50% EtOAc/MeOH then fractionated by RP HPLC (YMC-PAK ODS-AQ) with a gradient of 50% aqueous MeOH to 100% MeOH to yield 8 fractions. A 2008 collection (PSC08-08-A) of fresh algae was extracted with MeOH (4 L×3). The combined extract was concentrated and the residue was partitioned between CH₂Cl₂ and H₂O, Subsequently, the CH₂Cl₂ layer was concentrated in vacuo to give a dark green crude (PSC08-8-A-A, 6.5 g). The residue was subjected to Si gel column chromatography with a hexanes/EtOAc/MeOH gradient solvent system to give 12 fractions. Fractions 9 and 10 (PSC08-8-A-A-9, 414 mg, and PSC08-8-A-A-10, 178 mg) were identified as active fractions.
(ii) A process comprising seven steps for obtaining, purifying, or concentrating the active compound was developed (FIG. 6). Whole algae thalli where used for the preparation of protein extracts using the commercial kit P-PER (Pierce). 2) The aqueous phase of this extraction demonstrated antiviral activity. 3) Subsequently it was further fractionated using membrane filtration as follows: The aqueous phase was sequentially 4) filtered through a 30,000 Dalton Amicon membrane. What remains unfiltered is named retentate 1 and the material that passes through the filter is called filtrate 1. 5). Filtrate 1 was passed through a 10,000 Dalton Amicon filter resulting in retentate 2 and filtrate 2. 6). Filtrate 2 was passed through a 3000 Dalton Amicon filter producing retentate 3 and filtrate 3. 7). Retentate 1, 2 and 3 and filtrate 1 and 2 presented antiviral activity indicating that the active compound has a molecular size larger than 3,000 Daltons. One aspect of the current invention concerns the composition, and component compounds thereof, as prepared in (i) above. Advantageously, this composition (through the action of its component compounds) can inhibit, control, or destroy viruses, including influenza virus.

In one embodiment, the subject invention provides bioactive compounds (e.g., proteins) obtainable from the Gigartina extract. Also provided are compositions (including Gigartina extract) that comprise proteins of the subject invention. Advantageously, the bioactive compounds (e.g., proteins) of the subject invention have anti-viral effects (e.g., inhibiting, controlling, or destroying virus, including influenza viruses). In a specific embodiment, the proteins of the subject invention are glycosylated. In another specific embodiment, the anti-viral compound contained in the Gigartina extract is not a monosaccharide, disaccharide, or oligosaccharide, or polysaccharide (such as a sulfated polysaccharide).

In one embodiment, the anti-viral proteins of the subject invention comprise one or more amino acid sequences, selected from SEQ ID NO:1 to SEQ ID NO:18, or fragments thereof exhibiting anti-viral activity. In another embodiment, a protein of the subject invention has amino acid substitution of, deletion from, and/or insertion into a sequence selected from SEQ ID NO:1 to SEQ ID NO:18; wherein a total of no more than 1, 2, 3, 4, or 5 amino acids are substituted, deleted, and/or inserted, and wherein the protein has anti-viral effect. In a specific embodiment, the peptide fragment has, for example, at least 7, 8, 9, 10, 11, 12, 13, 14, 15, 16, 17, 18, 19, 20, 21, 22, 23, 24, or 25 contiguous amino acids of its corresponding sequence selected from SEQ ID NOs:1-18.

Desired amino acid substitutions (whether conservative or non-conservative) can be determined by those skilled in the art at the time such substitutions are desired. In certain embodiments, conservative amino acid substitutions also encompass non-naturally occurring amino acid residues which are typically incorporated by chemical peptide synthesis rather than by synthesis in biological systems.

Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form.

In a further embodiment, the subject invention provides nucleic acid probe molecules comprising a sequence that encodes a peptide sequence selected from SEQ ID NOs: 1-18 or a fragment thereof. Such a nucleic acid molecule can be used as an oligoncleotide or polynucleotide probe. Also provided are methods for producing the anti-viral Gigartina proteins of the subject invention, wherein the Gigartina protein comprises one or more amino acid sequences selected from SEQ ID NO:1 to SEQ ID NO:18 or a fragment thereof.

In one embodiment, the subject invention provides a method for producing the anti-viral protein comprising one or more amino acid sequences selected from SEQ ID NO:1 to SEQ ID NO:18, or a fragment thereof, wherein the method comprises the following steps:

a) providing a library of candidate nucleic acid molecules that are extracted from a Gigartina species;

b) hybridize the library of candidate nucleic acid molecules extracted from the Gigartina species to nucleic acid probe molecules that encode one or more peptide sequences under high stringency conditions, wherein the peptide sequence is selected from SEQ ID NO:1 to SEQ ID NO:18, or a fragment thereof;

c) selecting the candidate nucleic acid molecule if said molecule hybridizes to a nucleic acid probe molecule;

d) expressing the selected nucleic acid molecule to obtain a Gigartina protein molecule; and

e) testing the anti-viral activity of the Gigartina protein molecule and selecting the protein Gigartina molecule that exhibits anti-viral activity.

The nucleic acid molecules of the subject invention encompass DNA molecules (e.g. genomic DNA and cDNA) and RNA molecules. In addition, the subject nucleic acid molecules may be single-stranded or double-stranded. The subject nucleic acid molecules may also artificially created (e.g. recombinant DNA and chemically-synthesized polynucleotide molecules).

“Hybridization” refers to a reaction in which one or more polynucleotides react to form a complex that is stabilized via hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. Various degrees of stringency of hybridization can be employed. The more severe the conditions, the greater the complementarity that is required for duplex formation. Severity of conditions can be controlled by temperature, probe concentration, probe length, ionic strength, time, and the like.

Preferably, hybridization is conducted under high stringency conditions by techniques well known in the art, as described, for example, in Keller, G. H. & M. M. Manak, DNA Probes, and the companion volume DNA Probes: Background, Applications, Procedures (various editions, including 2^{nd} Edition, Nature Publishing Group, 1993). Hybridization is also described extensively in the Molecular Cloning manuals published by Cold Spring Harbor Laboratory Press, including Sambrook & Russell, Molecular Cloning: A Laboratory Manual (2001).

A non-limiting example of high stringency conditions for hybridization is at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C. A non-limiting example of hybridization conditions are conditions selected to be about 1, 2, 3, 4, 5, 6, 7, 8, 9, 10, 11, 12, 13, 14, 15, 16, 17, 18, 19, 20, 21, 22, 23, 24, or 25° C. lower than the thermal melting point (Tₘ) for the specific sequence in the particular solution.

Tₘ is the temperature (dependent upon ionic strength and pH) at which 50% of the target sequence hybridizes to a perfectly matched probe. Tₘ typically increases with [Na⁺] concentration because the sodium cations electrostatically shield the anionic phosphate groups of the nucleotides and minimize their repulsion. The washes employed may be for about 5, 10, 15, 20, 25, 30, or more minutes each, and may be of increasing stringency if desired.

Calculations for estimating Tₘ are well-known in the art. For example, the melting temperature may be described by the following formula (Beltz, G. A., K. A. Jacobs, T. H. Eickbush, P. T. Cherbas, and F. C. Kafatos, Methods of Enzymology, R. Wu, L. Grossman and K. Moldave [eds.] Academic Press, New York 100:266-285, 1983).

Tm=81.5° C.+16.6 Log [Na+]+0.41(%G+C)−0.61(% formamide)−600/length of duplex in base pairs.

A more accurate estimation of Tₘ may be obtained using nearest-neighbor models. Breslauer, et al., Proc. Natl. Acad. Sci. USA, 83:3746-3750 (1986); SantaLucia, Proc. Natl. Acad. Sci. USA, 95: 1460-1465 (1998); Allawi & SantaLucia, Biochemistry 36:10581-94 (1997); Sugimoto et al., Nucleic Acids Res., 24:4501-4505 (1996). Tₘ may also be routinely measured by differential scanning calorimetry (Duguid et al., Biophys J, 71:3350-60, 1996) in a chosen solution, or by other methods known in the art, such as UV-monitored melting. As the stringency of the hydridization conditions is increased, higher degrees of homology are obtained.

The anti-viral activity of the proteins of the subject invention can be determined using various techniques described in the subject application, such as for example, the primary screen, the secondary screen, the plaque reduction assay, and the virus progeny reduction assay. In certain embodiments, anti-viral proteins of the subject invention enables at least 5%, 10%, 15%, 20%, 25%, 30%, 35%, 40%, 45%, 50%, 60%, 70%, 80%, 90%, or 100% decrease in viral titers (e.g., TCID₅₀). In one embodiment, the subject invention provides isolated or purified components (e.g., compounds, proteins).

As used herein, “isolated” refers to components that have been removed from any environment in which they may exist in nature. For example, an isolated protein would not refer to the compound as it exists in a Gigartina species. In preferred embodiments, the isolated or purified component (e.g., compounds, proteins) of the subject invention are at least 75% pure, preferably at least 90% pure, more preferably are more than 95% pure, and most preferably are more than 99% pure (substantially pure).

In accordance with the subject invention, methods for inhibiting, controlling, or destroying viruses in a host include contacting virally-infected cells with an effective amount of the new pharmaceutical compositions of the invention. The viruses inhibited by the invention are those which are susceptible to the subject compounds described herein or compositions comprising those compounds.

The subject invention further provides methods of using compounds and compositions of the invention, e.g., methods of inhibiting, controlling, or destroying influenza virus, or other viruses, in an animal, preferably a mammal. Most preferably, the invention comprises a method for the antiviral treatment of a human in need of such treatment, i.e., a human hosting one or more viruses, including influenza virus, other types of virus, or virus that is resistant to a known antiviral therapy.

In one embodiment, the subject invention provides methods for prevention and/or treatment of viral infection including, but not limited to, influenza. In an embodiment, the method comprises administering, to a subject in need of such treatment, an effective amount of the compounds and/or compositions (e.g., Gigartina extract or Gigartina proteins) of the subject invention.

The term “influenza virus,” as used herein, includes any strain of influenza viruses that is capable of causing disease in an animal or human subject, or that is an interesting candidate for experimental analysis. Influenza viruses are described in Fields, B., et al., Fields' Virology, 4^{th} ed., Philadelphia: Lippincott Williams and Wilkins; ISBN: 0781718325, 2001 &/or in the Collier and Oxford, Human Virology, Third Edition, Oxford University Press, Oxford, England: ISBN 978-0-19-856660-1.

In one embodiment, the compositions and therapeutic methods of the subject invention are useful for preventing, treating, or ameliorating influenza including, but not limited to, influenza A and influenza B. In certain embodiments, the subject invention are useful for preventing, treating, or ameliorating influenza A including, but not limited to, infection by any of the strains of selected from H1N1, H1N2, H1N3, H1N4, H1N5, H1N6, H1N7, H1N8, H1N9, H2N1, H2N2, H2N3, H2N4, H2N5, H2N6, H2N7, H2N8, H2N9, H3N1, H3N2, H3N3, H3N4, H3N5, H3N6, H3N7, H3N8, H3N9, H4N1, H5N2, H5N3, H5N4, H5N5, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N6, H7N7, H7N8, H7N9, H8N1, H8N2, H8N3, H8N4, H8N5, H8N6, H8N7, H8N8, H8N9, H9N1, H9N2, H9N3, H9N4, H9N5, H9N6, H9N7, H9N8, H9N9, H10N1, H10N2, H10N3, H10N4, H10N5, H10N6, H10N7, H10N8, H10N9, H11N1, H11N2, H11N3, H11N4, H11N5, H11N6, H11N7, H11N8, H11N9, H12N1, H12N2, H12N3, H12N4, H12N5, H12N6, H12N7, H12N8, H12N9, H13N1, H13N2, H13N3, H13N4, H13N5, H13N6, H13N7, H13N8, H13N9, H14N1, H14N2, H14N3, H14N4, H14N5, H14N6, H14N7, H14N8, H14N9, H15N1, H15N2, H15N3, H15N4, H15N5, H15N6, H15N7, H15N8, H15N9, H16N1, H16N2, H16N3, H16N4, H16N5, H16N6, H16N7, H16N8, or H16N9.

In a specific embodiment, the compositions and therapeutic methods of the subject invention are useful for preventing, treating, or ameliorating infections caused by any of the strains selected from H1N1, H3N2, H5N1, or H7N1.

The term “treatment” or any grammatical variation thereof (e.g., treat, treating, and treatment etc.), as used herein, includes but is not limited to, ameliorating or alleviating a symptom of a disease or condition, reducing, suppressing, inhibiting, lessening, or affecting the progression, severity, and/or scope of a condition.

The term “prevention” or any grammatical variation thereof (e.g., prevent, preventing, and prevention etc.), as used herein, includes but is not limited to, delaying the onset of symptoms, preventing relapse to a disease, increasing latency between symptomatic episodes, or a combination thereof. Prevention, as used herein, does not require the complete absence of symptoms.

The term “subject,” as used herein, describes an organism, including mammals such as primates, to which treatment with the compositions according to the present invention can be provided. Mammalian species that can benefit from the disclosed methods of treatment include, but are not limited to, apes, chimpanzees, orangutans, humans, monkeys; and domesticated animals such as dogs, cats, horses, cattle, pigs, sheep, goats, chickens, mice, rats, guinea pigs, and hamsters.

In preferred embodiments of the invention, the extracts or compounds are substantially pure, i.e., contain at least 25%, 50%, 75%, 85%, 90%, 95%, or 99% of the biologically active extract(s) or compound(s) as determined by established analytical methods. In further preferred methods of the invention, salts within the scope of the invention are made by adding mineral acids, e.g., HCl, H₂SO₄, or strong organic acids, e.g., formic, oxalic, in appropriate amounts to form the acid addition salt of the parent compound or its derivative. Likewise, base addition salts may be appropriate for some compounds of the invention. Also, synthesis-type reactions may be used pursuant to known procedures to add or modify various groups in the preferred compounds to produce other compounds within the scope of the invention.

The scope of the invention is not limited by the specific examples and suggested procedures and uses related herein since modifications can be made within such scope from the information provided by this specification to those skilled in the art.

As used in this application, the terms “analogs,” refers to compounds which are substantially the same as another compound but which may have been modified by, for example, adding or removing side groups.

A more complete understanding of the invention can be obtained by reference to the following specific examples of compounds, compositions, and methods of the invention. The following examples illustrate procedures for practicing the invention. These examples should not be construed as limiting. All percentages are by weight and all solvent mixture proportions are by volume unless otherwise noted. It will be apparent to those skilled in the art that the examples involve use of materials and reagents that are commercially available from known sources, e.g., chemical supply houses, so no details are given respecting them.

### Example 1

### Isolation and Antiviral Screening of Marine Extracts

### A. Collection and Taxonomy of the Source Organism(s).

An extensive library of marine organisms including more than 2,500 specimens collected from diverse regions of the globe has previously been prepared. The various marine environments from which this collection was constructed encompass tropical coral reefs, temperate kelp forests, and polar communities. Macroorganisms and microorganisms have been collected; microbial isolates collected from the tissue of a macroorganism, a sediment sample, or water column have been preserved in various media with glycerol.

Collections have been made primarily by scuba divers, targeting approximately 1 kg samples that are separated into collection bags. Upon completion of the dive, samples have been immediately placed into ice-cooled chests. Samples are removed one at a time for further documentation, which includes extensive field notes, further photography of the specimen out of the water, and sub-sampling for organic extractions. Selected smaller whole invertebrates and algae or sub-samples of larger macro-organisms (4×1 g) for microbe-isolation studies have been collected in the field using aseptic technique. Samples for voucher specimens were pressed. Detailed information relating to the collection of certain relevant specimen(s) is as follows.

Collection of the rhodophyte Gigartina skottsbergii Setchell & Gardner 1936 (Phylum: Rhodophyta, Class: Florideophyceae, Sub Class: Rhodymeniophycidae, Order: Gigartinales, Family: Gigartinaceae) was done by hand during SCUBA dives within 3.5 km of Palmer Station on Anvers Island off the western Antarctic Peninsula (64° 46.5′ S, 64° 03.3′ W) at a depth of 5-12 m. Collections were made during three periods: early November to late December 2001, early March to early June 2007, and early January to mid March 2008.

### B. Methods of Preparation and Screening of Extracts

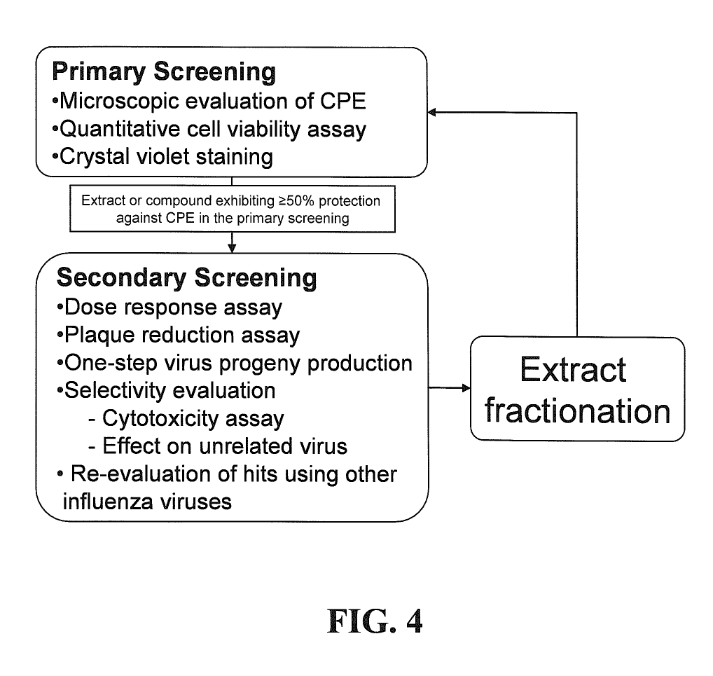

FIG. 4 illustrates schematically the screening pathway and procedures followed. The following paragraphs describe these procedures in greater detail.

### Preparation of Extracts

In one embodiment, a 2001 collection (PSC01-12) of frozen algae (2.2 kg) was extracted with CH₂Cl₂/MeOH (1:1, 1 L×3). The combined extract was concentrated to a dark green crude (3.6 g). The residue was subjected to Si gel column chromatography with a hexanes/EtOAc/MeOH gradient solvent system to give 13 fractions. Fraction 9 (PSC01-12-6-I, 309.3 mg) eluted with approximately 50% EtOAc/MeOH then was fractionated by RP HPLC (YMC-PAK ODS-AQ) with a gradient of 50% aqueous MeOH to 100% MeOH to yield 9 fractions. A 2007 collection (PSC07-52) of fresh algae (12.1 kg) was extracted with MeOH (4 L×3). The combined extract was concentrated (344.4 g), and the residue was partitioned between CH₂Cl₂ and H₂O, Subsequently, the CH₂Cl₂ layer was concentrated in vacuo to give a dark green crude (PSC07-52-A, 20.2 g). The residue was subjected to Si gel column chromatography with a hexanes/EtOAc/MeOH gradient solvent system to give 12 fractions. Fraction 9 (PSC07-52-A-I, 2.9 g) eluted with approximately 50% EtOAc/MeOH then fractionated by RP HPLC (YMC-PAK ODS-AQ) with a gradient of 50% aqueous MeOH to 100% MeOH to yield 8 fractions. A 2008 collection (PSC08-08-A) of fresh algae was extracted with MeOH (4 L×3). The combined extract was concentrated and the residue was partitioned between CH₂Cl₂ and H₂O, Subsequently, the CH₂Cl₂ layer was concentrated in vacuo to give a dark green crude (PSC08-8-A-A, 6.5 g). The residue was subjected to Si gel column chromatography with a hexanes/EtOAc/MeOH gradient solvent system to give 12 fractions. Fractions 9 and 10 (PSC08-8-A-A-9, 414 mg, and PSC08-8-A-A-10, 178 mg) were identified as active fractions.

In another embodiment, a process comprising the following steps for obtaining, purifying, or concentrating the active compound was developed (FIG. 6A). Whole algae thalli were used for the preparation of protein extracts using the commercial kit P-PER (Pierce). 2) The aqueous phase of this extraction demonstrated antiviral activity. 3) Subsequently it was further fractionated using membrane filtration as follows: The aqueous phase was sequentially 4) filtered through 30,000 Dalton Amicon membrane. What remains unfiltered is named retentate 1 and the material that passes through the filter is called filtrate 1. 5) Filtrate 1 was passed through a 10,000 Dalton Amicon filter resulting in retentate 2 and filtrate 2. 6) Filtrate 2 was passed through a 3000 Dalton Amicon filter producing retentate 3 and filtrate 3. 7) Retentate 1, 2 and 3 and filtrate 1 and 2 presented antiviral activity indicating that the active compound has a molecular size larger than 3,000 Daltons. In a specific embodiment, the anti-viral compounds contained in the Gigartina extract include proteins and/or glycosylated proteins.

### Cells and Viruses

Cells cultures used in screening are Madin Darby canine kidney (MDCK) cells, obtained from American Type Culture Collection (Manassas, Va., CCL-34, passage 55) and grown in Eagle minimum essential medium (MEM, Invitrogen) with 10% reconstituted fetal calf serum (HyClone III). The cells are trypsinized and then resuspended at 3×10⁵ cells/mL in assay media (for Secondary screening DMEM, high glucose without phenol red) supplemented with gentamicin and 0.5% BSA for all subsequent steps. Cells are plated manually and incubated at 37° C. and 5.0% CO₂ for 24 h prior to virus addition.

Influenza virus stocks are prepared by growing influenza strain A/PR8/38 (H1N1), A/Wyoming/3/2003 (H3N2) and B/Lee/40 in MDCK cells. The supernatant from infected MDCK cells is serially diluted and used for isolation of a single plaque. A single plaque from second round of plaque purification is selected and resuspended in serum-free Dulbecco's modified Eagle's medium (DMEM, Invitrogen, Carlsbad, Calif.) containing 0.35% bovine serum albumin (BSA, Invitrogen, Fraction V). The plaque-purified virus is used to inoculate three T150 flaks containing MDCK cells (see below) at a multiplicity of infection of 0.001 PFU/cell. The supernatant is collected 72 h post infection, aliquot and stored a −80° C. until needed.

Multiplicity of infection is determined via ninety-six well plates that are plated with MDCK cells at a density of 1.5×10⁴ perwell (3×10⁵ cells/mL, 50 μl of cells/well). Twenty four hours after plating, the media is replaced with MEM containing 50 μl of N-acetyl trypsin (5 μg/mL, diluted in assay media). Amplified influenza virus is diluted 100-fold in assay media containing 2.5 μg/mL N-acetyl trypsin, then added to the first column of the plate and successively serially diluted across the remaining plate columns. Fresh pipette tips are used for each dilution to avoid virus carry over to subsequent columns, and the cells in the last plate column is left uninfected as controls. The plates are incubated at 37 C/5.0% CO₂ for 72 h. Control wells containing medium without cells are used to obtain a value for background absorbance. After incubation at 37° C. for 72 h the plates are visually scored and analyzed using CellTiter 96® AQueous One Solution as indicated below. Three replicate plates are analyzed; individual plates are averaged to establish the TCID₅₀ and determine the virus dilution needed to obtain the appropriate MOI for each viral strain.

### Primary Screen

Bacterial extract stocks are prepared at a concentration of 60 mg/mL dissolved in 100% DMSO. DMSO stocks are stored at room temperature for 2-3 weeks in the dark. The extracts are pre-diluted to 600 μg/mL in DMEM supplemented with 0.5% BSA, thus reducing the DMSO to 1%. Although it is not anticipated to find solubility problems (absence of terpenoids) in these bacterial extracts, if such occurs, the highest soluble concentration is tested.

Primary screening of marine extracts includes microscopic evaluation of cytopathic effect (CPE). The primary screening is performed using influenza virus strain A/Wyoming/03/2003 (H3N2). The primary screening proposed is based on the determination of reduction in CPE as evaluated using visual scoring. Each well is observed at a magnification of 40× using an inverted microscope. Complete CPE is recorded with two plus signs (++), partial CPE (some cells appear without signs of CPE) is recorded with one plus sign (+), complete protection (no signs of CPE are observable) is recorded with a minus sign (−).

Cell viability is quantified using a commercially available MTT cell viability test (CellTiter 96® AQueous One Solution, Promega). This colorimetric method is used also in the secondary screening for the determination of dose response and cytotoxic effects. This approach has been previously validated and confirmed to be statistically comparable to other methods.

A single-dose (100 μg/mL), single-well per extract screening is conducted in 96-well plates. Briefly, 50 μl of media (DMEM/F12(1:1). Hyclone SH30272.01, supplemented with 0.35% BSA and 2.5 μg/mL of N-Acethyl trypsin, and sodium pyruvate) is added to each well, followed by addition of 20 μl of extract (600 μg/mL) to each test well. The virus is added in 50 μl volume at a dilution that produces CPE in 99% of the wells corresponding to approximately 40 TCID₅₀(1×10⁻⁴ dilution of the virus stock of 7.8×10⁶ TCID₅₀/mL). Subsequently, 50 μl of the above media containing 16,000 MDCK (NBL-1, ATCC Number CCL-22) is added to each well. The final volume in each well is 120 μl. Plates are then incubated at 37° C., in 5% CO₂, for 72 h. The preparation of the master and mother plates and the handling of media, marine extracts, virus and cells is performed employing a Biomek 3000 and BC NX robots placed inside a biosafety level 2 cabinet. Experimental controls in each plate include uninfected cells, infected cells, and ribavirin at a concentration of 5 μg/mL. Reduction of CPE is qualitatively evaluated by direct observation of cytopathic effect using an inverted light microscope. After the visual evaluation 20 μl of CellTiter 96 Aqueous-One reagent is added to each well, mixed by vortexing and incubated at 37° C. for 2 h. Optical density is measured at absorbance of 490 nm using a BioTek Synergy HT plate reader. Percentage of protection is calculated using the following formula: (1−((μ_{c}−OD of Sample)/(μ_{c}−μᵥ)))*100, where μ_{c} is the mean optical density (OD) value of the uninfected cells and μᵥ is the mean OD value of the infected cells.

Crystal violet staining is conducted after measurement of the cell viability. The plates are stained using a 2.5% crystal violet solution in PBS containing 4% formaldehyde. The purpose of performing this staining is to create a permanent record of the plates and to corroborate the cell viability assay with the visual scoring of CPE. To confirm the results of primary screening, promising extracts (those displaying ≧50% protection against CPE at 100 μg/mL) are re-tested in triplicate using the primary screening protocol.

### Secondary Screen

Secondary screening is conducted on extracts identified as being active in the primary screen. Active extracts or compounds are those that display ≧50% protection against CPE at the 100 μg/mL dose of the primary screen.

Secondary screening is carried out with four assays employing influenza A/Wyoming/03/2003 (H3N2). These assays include dose dependant response evaluation, plaque reduction assay, virus progeny reduction, and assessment of selectivity by evaluating the cytotoxicity in mammalian cells.

Other viruses can also be employed in secondary screening. Extracts are evaluated for their spectrum of inhibition by testing of additional viruses including A/NWS/33 (H1N1), B/Lee/40, and the low pathogenic avian influenza viruses A/TY/WI/68 (H5N9) and A/TY/UT/24721-10/95 (H7N3). Extracts are evaluated for specificity by observing the effect on the growth of unrelated viruses (cytopathic bovine viral diarrhea virus, Singer strain).

dose dependent response

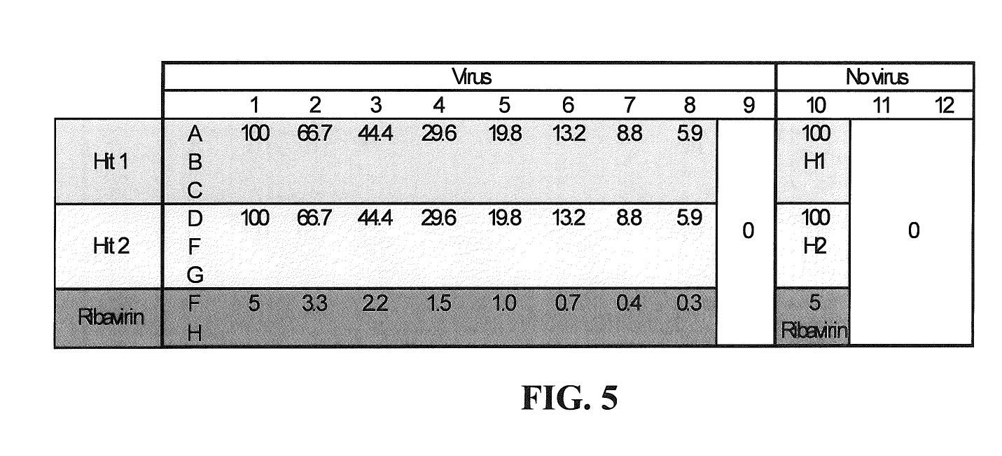

To assess whether active extracts identified during the primary screening cause a quantitatively measurable dose response, extracts are serially diluted 2/3-fold over 8 different concentrations (FIG. 5) using a Biomek 3000 (Beckman, Fullerton, Calif.) and the percentage protection is determined using the same approach that the one described for the primary screening. The quantitative analysis is performed using the cell viability assay previously described (CellTiter 96® AQueous One Solution, Promega).

Concentration response data is analyzed by a nonlinear regression logistic dose response model and the 50% and 90% inhibitory concentrations (IC₅₀s and IC₉₀s) for each compound will be calculated.

Each of the crude extracts are fractionated into 10-15 sub-fractions. For each crude extract and the associated sub-fractions, IC₅₀s and IC₉₀s are determined as outlined above. All crude and sub-fractions of a particular marine organism are assayed simultaneously (within one assay) and include ribavirin as reference drugs. For this phase we only use the A/WY/03/2003 virus. Extracts and sub-fractions with excellent activity and selectivity are further fractionated and assessed for activity in an iterative process. The most promising extracts and pure compounds are assessed for activity against a panel of influenza viruses which preferably include H5N1 as well as selected adamantine resistant strains.

### Plaque Reduction Assay

The effect of active marine extract is evaluated in plaque reduction assay. Briefly, 80% confluent MDCK cell monolayers in six-well plates are infected with 150 and 1500 pfu per well and incubated at 4° C. for 1 h to synchronize the infection. After this incubation period the unattached virus is removed by washing the cell monolayer with culture media. A semisolid agar overlay containing 2.5 μg/mL of N-Acetyl trypsin and 0.35% of BSA with or without active marine extract is used to cover the infected cell monolayer. After incubation at 37° C. for 72 h the monoloyers are fixed in situ using crystal violet solution for 2 h after which the agar overlay is removed and discarded. The size and number of plaques is quantified and compared to untreated controls.

### Virus Progeny Reduction

The virus progeny of wells exhibiting extract-induced CPE protection is also analyzed to quantitatively determine the reduction in virus progeny after a single replication cycle using TCID₅₀. Forty-eight well plates containing 80% confluent MDCK cell monolayers are infected with 40 TCID₅₀ of influenza virus in 600 μl of media containing N-Acetyl trypsin and BSA as previously indicated, and incubated at 37° C. for 24 h. The plates will be freeze-thaw three times and the media-cell suspension transferred to microcentrifuge tubes to pellet the cell debris. One hundred microliters of supernatant is diluted in 1/100. This dilution is added to the first eight wells of a 96-well tissue culture plate containing MDCK cells as described in previous sections. Subsequently the virus is diluted in a 10-fold serial dilution, and the CPE visually scored and the cell viability quantified by OD in the manner already described.

### Cytotoxicity Evaluation

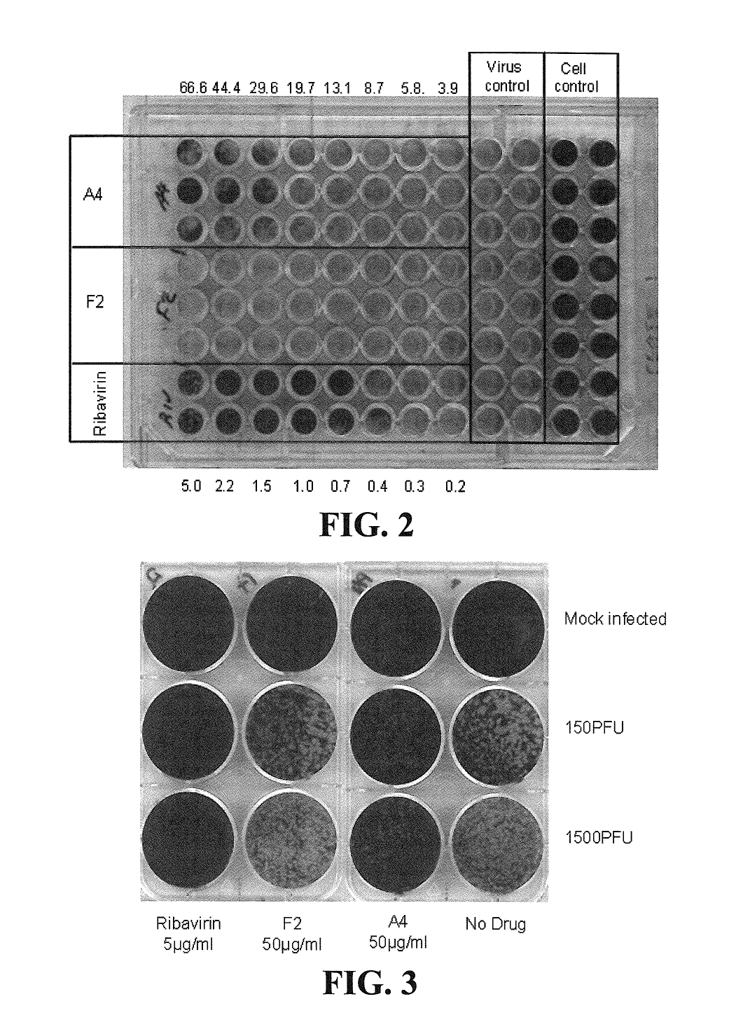

The selectivity of active marine extracts is evaluated using the same plate configuration described in FIG. 2, however it is preferable to use the cell line A549 in addition to MDCK at lower density since the latter are reportedly less susceptible to cytotoxic effect. The cytotoxic concentration 50% (CC50) is only evaluated after extract fractionation, however this approach serves to determine whether the cells are affected by the extract and therefore may be the cause responsible for the antiviral effect. The cells are plated at lower density (50% confluency) to aid in the evaluation of potential cytostatic effect. Ribavirin at 10 μg/mL and amantadine at 120 μg/mL are used as cytostatic and cytotoxic control drugs. The quantification of cell viability is measured using the cell viability assay previously described in the primary screening (CellTiter 96® AQueous One Solution, Promega).

### C. Results

Primary screening encompassing evaluation of CPE by microscope, determination of cell viability via OD measurements, and staining with crystal violet is conducted according to the methods as previously described. Extracts are screened in 96-well plates in a single-dose (100 μg/mL), single-well per extract format as indicated above.

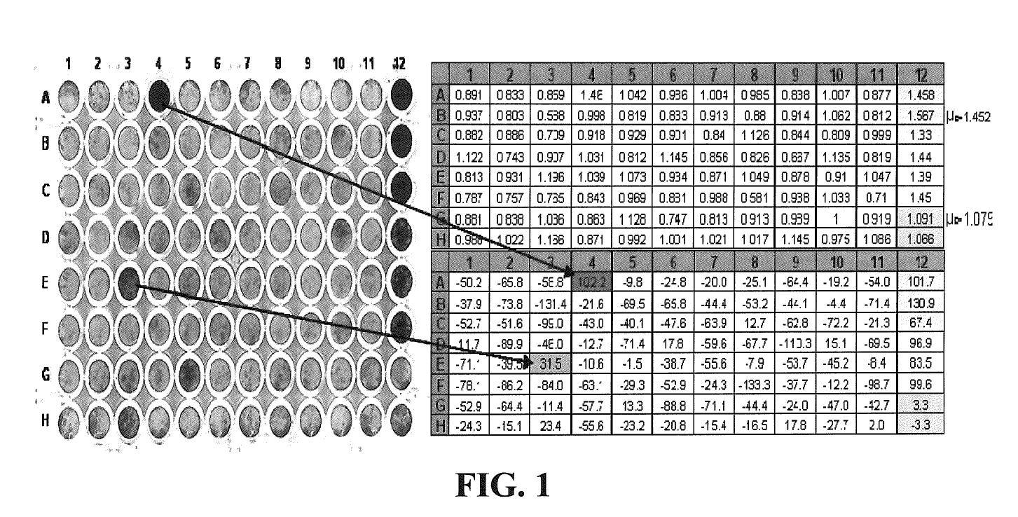

FIG. 1 presents a representative plate obtained during the primary screen. The extract in position A4 does not present CPE when observed under the microscope and the cell viability assay indicates complete protection at the 100 μg/mL extract dose. In contrast, the extract in position E3 presents CPE, but with a visible increase in cell protection. The partial protection observed for this well in the CPE assessment is in agreement with the quantitative cell viability assay (˜30% protection) determined by OD. Wells A-C12 contain uninfected control, D-F12 contains control drug ribavirin at 5 μg/mL and G12 and H12 are the virus infected control. It is noted that the crystal violet staining intensity that is visible in FIG. 1 is not the principal quantitative measure of cell protection, but rather is used as an additional indicator of cell protection. Using this primary screening approach, 648 extracts are evaluated and 5 extracts are identified that produce a level of protection of ≧50%, resulting in a hit rate of approximately 0.7%.

Secondary screening of compounds that induce ≧50% protection at 100 μg/mL is initiated, resulting in the further characterization of extract A4 identified in the primary screen. Extract A4 is evaluated using a series of eight 2/3 serial dilutions to determine whether this extract results in protection against influenza virus infections in a dose dependant manner. The resulting concentrations in μg/mL are 66.6, 44.4, 29.6, 19.7, 13.1, 8.7, 5.8, and 3.9. Percentage of protection is quantified using the previously mentioned cell viability assay. FIG. 2 presents the results of this evaluation. F2 is an inactive extract. It is seen that extract A4 and extract F2 are tested in triplicate at each concentration. Ribavirin is used as drug control at concentrations of 5 to 0.2 μg/mL as shown.

The ability of the A4 and F2 extracts to inhibit the growth of the virus in multiple rounds of replication is tested using the plaque reduction assay. For these experiments 6-well plates containing 80% confluent MDCK cell monolayers are inoculated with the indicated pfu as shown in FIG. 3. The plates are then incubated for 1 h at 37° C. before adding a semisolid agar overlay containing 50 μg/mL of marine extracts A4 and F2. Ribavirin is used as drug control at 5 μg/mL. The plates are incubated at 37° C. for 72 h and then stained using crystal violet/formalin solution. An uninfected well containing 50 μg/mL of the marine extract A4 is included to evaluate the toxicity of the compound. Extract A4 induces the formation of smaller plaques than the untreated or F2 extract controls. Ribavirin completely inhibits the formation of plaques at the indicated dose. Using this approach we have identified three distinct extracts with antiviral activity.

Each marine bacterial extract is estimated to contain an average of 30 compounds; invertebrate and algal extracts are expected to be equally complex. Therefore 100 μg/mL results in an approximate concentration of ˜3 μg/mL per compound. This concentration is within the range used for screening of chemical libraries for identification of antiviral leads. As illustrated by the results above, a large number of extracts can be rapidly screened for antiviral activity.

This selectivity screen has a number of advantages, particularly in identifying anti-influenza-selective extracts. Furthermore, this cell based screen offers the additional advantage of evaluating inhibitory activity of multiple molecular targets and viral stages of replication and cytotoxicity of extracts simultaneously.

### Example 2

### Activity-Guided Fractionation of Marine Extracts

As employed herein, activity-guided fractionation refers generally to any known means of fractionating a mixture into component parts (including chromatography, electrophoresis, extraction, sublimation, evaporation, dehydration, centrifugation, and other methods known in the art but too numerous to list) coupled with selecting a fraction that contains the desired activity (such as antiviral activity). The fractionation is “activity-guided” if the selection of a fraction is based directly on the results of an activity assay, or if the selection of a fraction is based on a property that is correlated with activity (for example, structure is a chemical property that may be correlated with activity). Fractions are subjected to primary and secondary screening as described previously, thereby allowing the identification of those fractions containing antiviral activity.

Extract A4 was identified in the primary screen as coming from a 2001 collection (PSC01-12) of Gigartina skottsbergii. The activity-guided fractionation was initiated by extraction of the frozen algae (2.2 kg) with CH₂Cl₂/MeOH (1:1, 1 L×3) to yield a dark green crude residue (3.6 g). The residue was subjected to Si gel column chromatography with a hexanes/EtOAc/MeOH gradient solvent system to give 13 fractions, from which fraction 9 was identified as the most active fraction. Fraction 9 (PSC01-12-6-I, 309.3 mg) eluted with approximately 50% EtOAc/MeOH then was fractionated by RP HPLC (YMC-PAK ODS-AQ) with a gradient of 50% aqueous MeOH to 100% MeOH to yield 9 fractions. A 2007 collection (PSC07-52) of fresh algae (12.1 kg) was extracted with MeOH (4 L×3). The combined extract was concentrated (344.4 g), and the residue was partitioned between CH₂Cl₂ and H₂O, Subsequently, the CH₂Cl₂ layer was concentrated in vacuo to give a dark green crude (PSC07-52-A, 20.2 g). The residue was subjected to Si gel column chromatography with a hexanes/EtOAc/MeOH gradient solvent system to give 12 fractions, from which fraction 9 was identified as the most active fraction. Fraction 9 (PSC07-52-A-I, 2.9 g) eluted with approximately 50% EtOAc/MeOH then fractionated by RP HPLC (YMC-PAK ODS-AQ) with a gradient of 50% aqueous MeOH to 100% MeOH to yield 8 fractions. A 2008 collection (PSC08-08-A) of fresh algae was extracted with MeOH (4 L×3). The combined extract was concentrated and the residue was partitioned between CH₂Cl₂ and H₂O, Subsequently, the CH₂Cl₂ layer was concentrated in vacuo to give a dark green crude (PSC08-8-A-A, 6.5 g). The residue was subjected to Si gel column chromatography with a Hexanes/EtOAc/MeOH gradient solvent system to give 12 fractions. Fractions 9 and 10 (PSC08-8-A-A-9, 414 mg, and PSC08-8-A-A-10, 178 mg) were identified as active fractions.

### Example 3

### Identification of Anti-Viral Gigartina Proteins

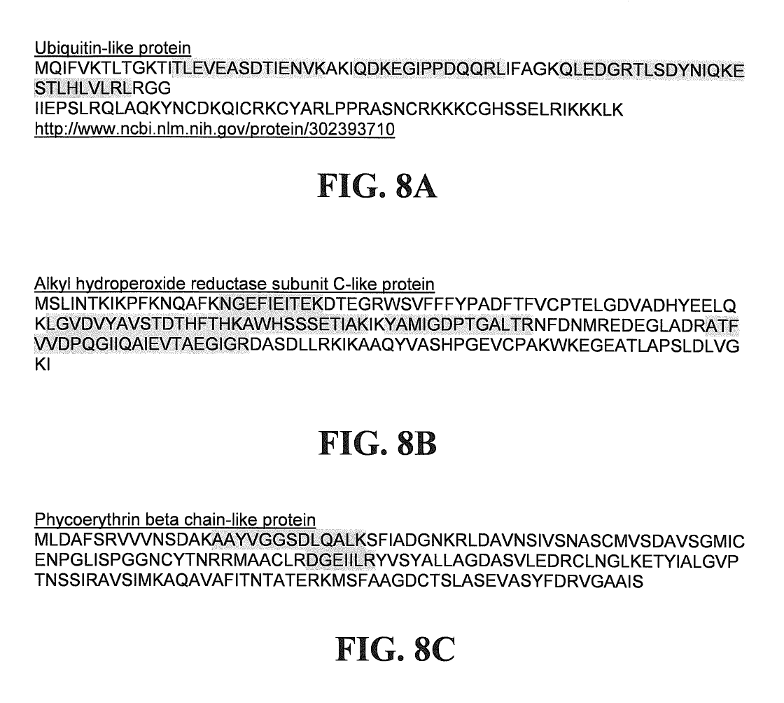

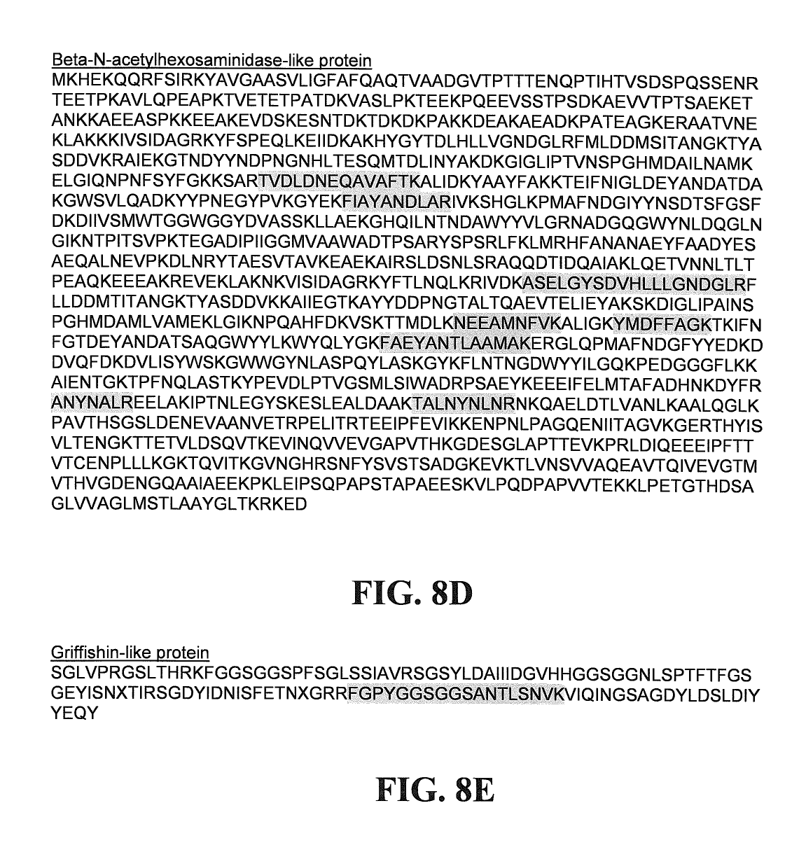

The anti-viral Gigartina Skottsbergii extract was analyzed using mass spectrometry, nuclear magnetic resonance, and ultrafiltration. The results indicate that protein components of the extract have anti-viral activity. Proteins contained in the Gigartina extract comprise one or more amino acid sequences selected from SEQ ID NO:1 to SEQ ID NO:18. As shown in FIGS. 8A-E, proteins contained in the Gigartina extract comprise one or more amino acid sequences that are also part of an ubiquitin-like protein, an alkyl hydroperoxide reductase subunit C-like protein, a phycoerythrin beta chain-like protein, a beta-N-acetylhexosaminidase-like protein, and/or a Griffishin-like protein. This suggests that the Gigartina extract can comprise ubiquitin-like proteins, alkyl hydroperoxide reductase subunit C-like proteins, phycoerythrin beta chain-like proteins, beta-N-acetylhexosaminidase-like proteins, and/or Griffishin-like proteins.

### Ubiquitin-Like Protein

The proteins contained in the Gigartina Skottsbergii extract were treated with an enzymatic deglycosilation cocktail. Deglycosilation facilitates subsequent tryptic digestion and determination of peptide sequences. A dominant protein band obtained by SDS-PAGE separation of the active Gigartina Skottsbergii extract was analyzed using peptide mapping of a tryptic digest (FIG. 6B) and was found to be homologous to a ubiquitin-like protein (FIG. 8A).

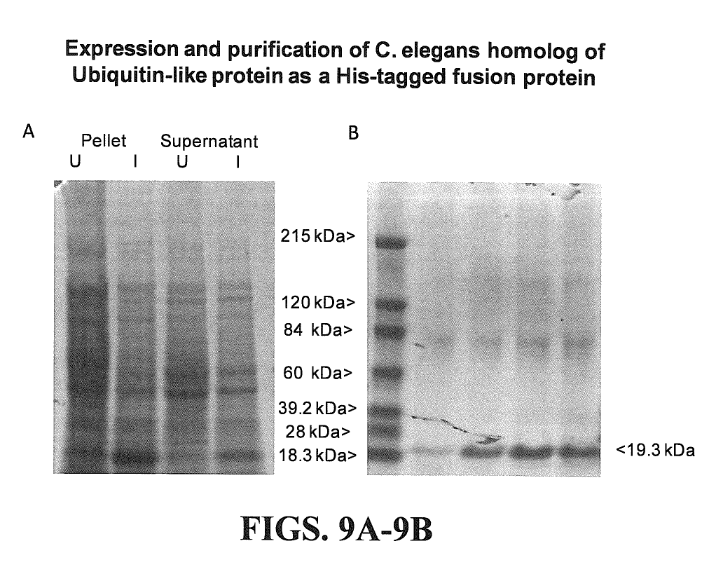

The purified protein band isolated from the Gigartina Skottsbergii extract has anti-viral activity, exhibiting an EC₅₀ of 4.3 μg/ml against influenza. Using the ubiquitin-like protein sequences (SEQ ID NOs:1-3), a cDNA copy based on the C. elegans homolog was cloned in a pET/LIC vector and used for expression of His-tagged recombinant protein in E. coli. Following purification, the protein was separated using SDS-PAGE (FIG. 9).

Sequences derived from the peptide mapping were used to create an artificial gene homolog of the ubiquitin-like protein, cloned as a fusion protein and codon optimized for expression in E. coli. The expression was tested at (FIG. 9A) 37° C. for 4 hs and the soluble supernatant (FIG. 9B) purified using IMACs. The insoluble (pellet) and soluble (supernatant) fractions and the purified proteins were separated using SDS-PAGE.

This homologous recombinant protein retained a moderate anti-influenza activity ˜25 μg/ml. The results indicate that: 1) the ubiquitin-like protein component contained in Gigartina skottsbergii is at least one of the anti-influenza components of the extract; and 2) anti-viral activity resides chiefly in the protein structure and not in post-translational modifications (e.g. glycosylation), since anti-viral activity is absent in proteins expressed by E. coli.

### Griffithsin-Like Protein

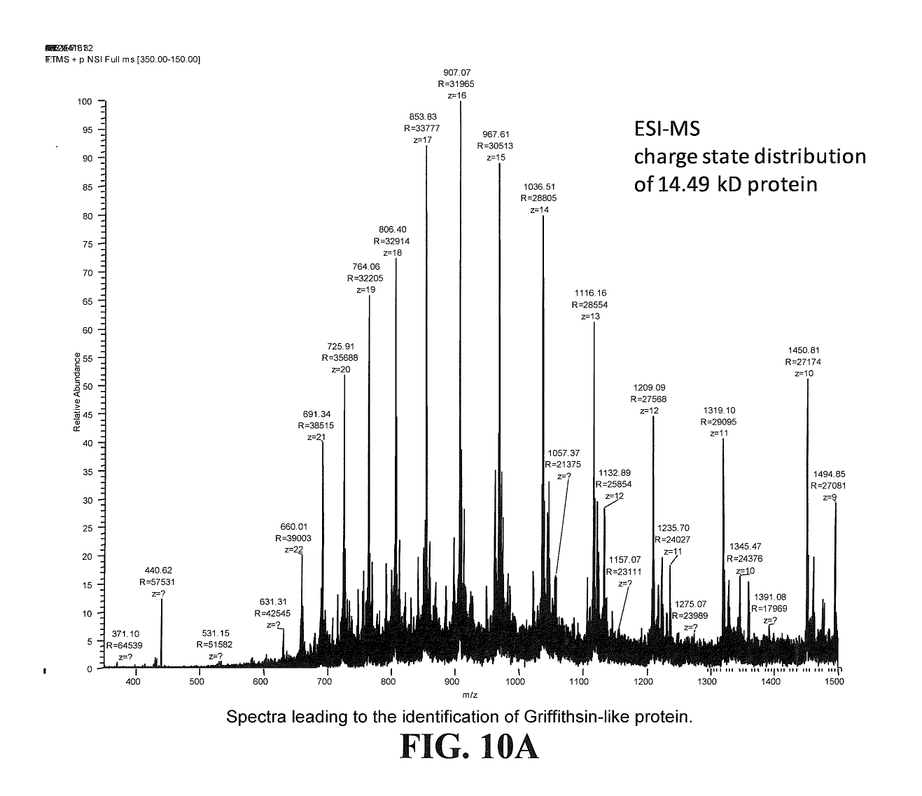

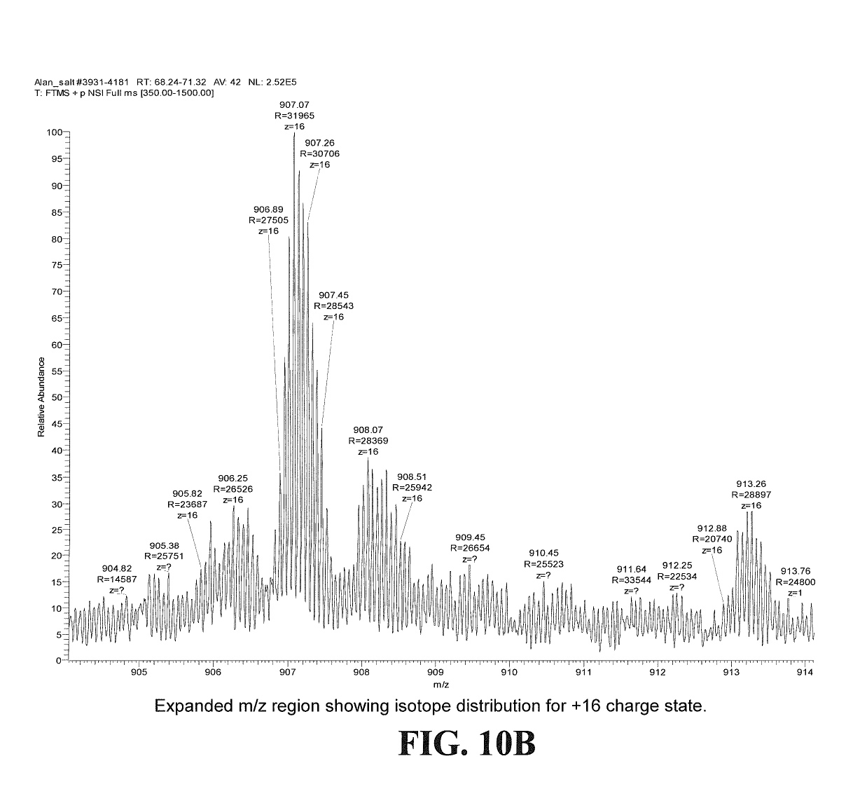

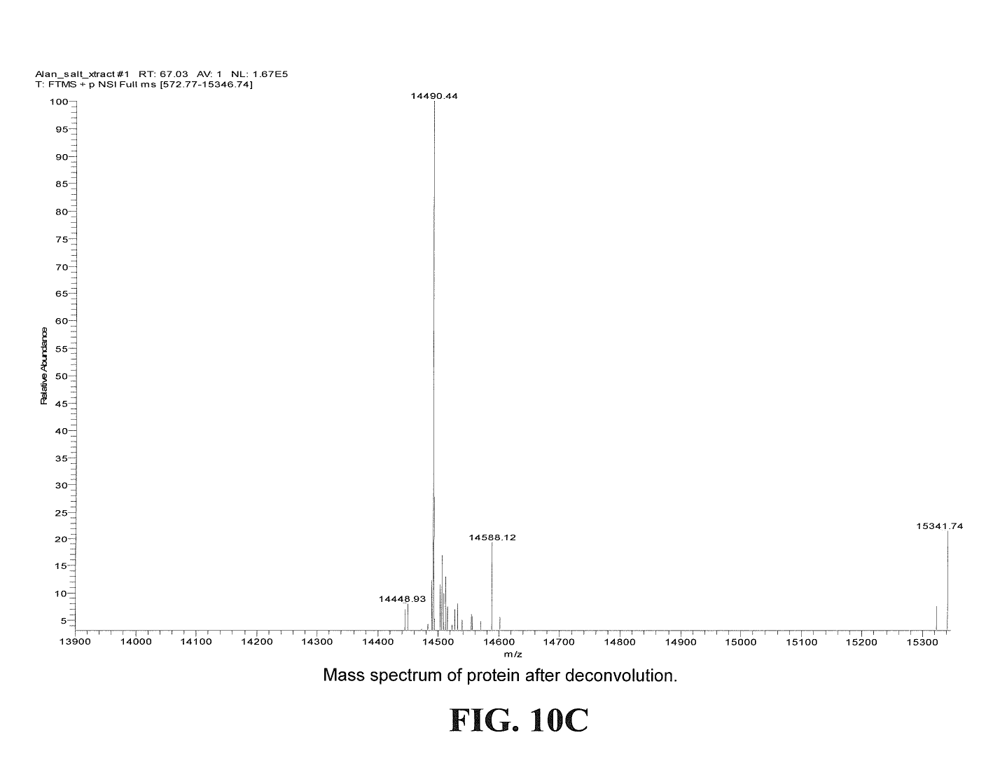

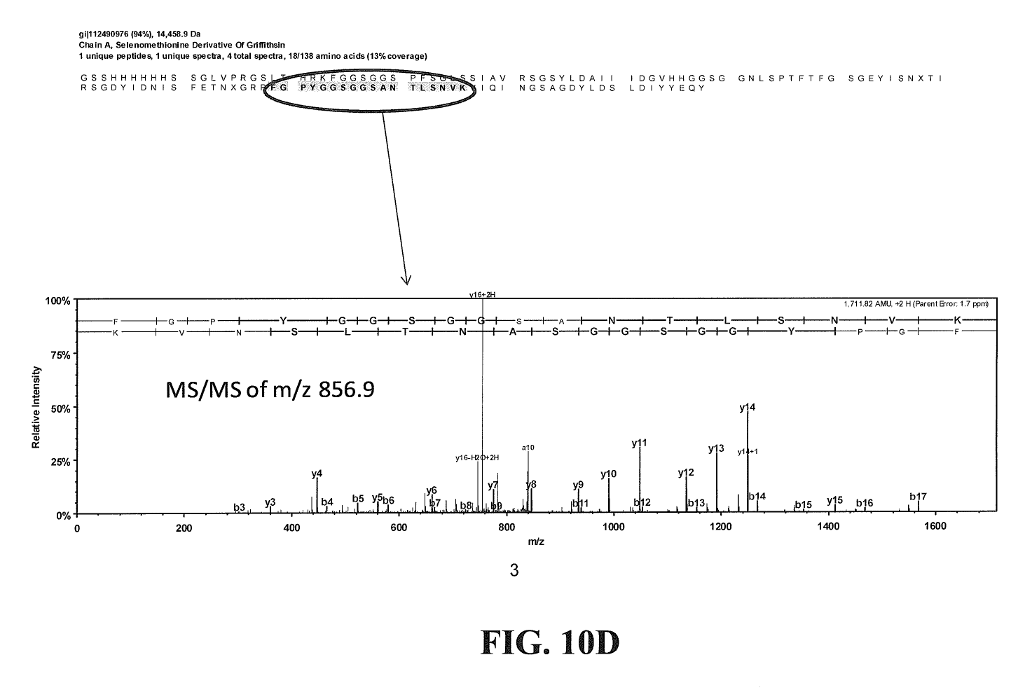

In addition, HPLC separation of the purified Gigartina proteinaceous fraction was subjected to peptide mapping, leading to the identification of an 18 amino acid signature sequence—a Griffithsin-like homolog, which is also present in Gigartina species such as Gigartina skottsbergii. FIG. 10 shows spectra of the Griffithsin-like protein present in the Gigartina skottsbergii extract.

### Example 4

### Formulation and Administration

The extracts of the invention, and fractions and components of the extracts, are useful for various non-therapeutic and therapeutic purposes. It is apparent from the testing that the compositions of the invention are effective for inhibiting, controlling, or destroying viruses. Because of the antiviral properties of the compounds, they are useful to prevent unwanted viral proliferation in a wide variety of settings including in vitro uses. They are also useful as standards and for teaching demonstrations. Further, they are also useful prophylactically and therapeutically for treating viral afflictions in animals and humans.

Therapeutic application of the new extracts, fractions, or components of the extracts, and compositions containing the extracts, fractions, or components can be accomplished by any suitable therapeutic method and technique presently or prospectively known to those skilled in the art. Further, the extracts and compounds of the invention have use as starting materials or intermediates for the preparation of other useful compounds and compositions.

The dosage administration to a host in the above indications is dependent upon the identity of the virus, the type of host involved, its age, weight, health, kind of concurrent treatment, if any, frequency of treatment, and therapeutic ratio.

The compounds of the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention are formulated such that an effective amount of the bioactive compound(s) or extract(s) is combined with a suitable carrier in order to facilitate effective administration of the composition.

In accordance with the invention, certain embodiments are compositions that comprise, as an active ingredient, an effective amount of one or more of the new extracts, fractions, or compounds, and that further comprise one or more non-toxic, pharmaceutically acceptable carriers or diluents. Examples of such carriers for use in the invention include ethanol, dimethyl sulfoxide, glycerol, silica, alumina, starch, and other carriers and diluents recognized in the art.

To provide for the administration of such dosages for the desired treatment, new compositions of the invention advantageously comprise between about 0.1% and 45%, and especially, 1 and 15%, by weight of the total of one or more of the new extracts, fractions, or compounds, relative to the weight of the total composition including carrier or diluent. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

It should be understood that the examples and embodiments described herein are for illustrative purposes only and that various modifications or changes in light thereof will be suggested to persons skilled in the art and are to be included within the spirit and purview of this application and the scope of the appended claims.

Each reference cited and clearly identified anywhere in this specification, including each publication, application, and patent, is incorporated by reference herein in its entirety.

We claim:

1. A composition comprising an extract of a Gigartina species, wherein said extract comprises a component having a molecular weight greater than 3,000 Daltons, and wherein said extract exhibits one or more anti-viral properties.

2. The composition of claim 1, wherein the Gigartina species is selected from the group consisting of Gigartina skottsbergii Setchell & Gardner 1936, Gigartina intermedia, Gigartina exasparata, Gigartina acicularis, Gigartina pistillata, Gigartina radula, Gigartina stellata, and Gigartina acicularis.

3. The composition of claim 1, wherein said extract inhibits viral growth.

4. The composition of claim 1, wherein said extract inhibits influenza replication.

5. The composition of claim 1, wherein said component comprises a protein.

6. The composition of claim 5, wherein said protein comprises:
1) an amino acid sequence selected from SEQ ID NO:1 to SEQ ID NO:18; or
2) an amino acid sequence that has amino acid substitution of, deletion from, and/or insertion into a sequence selected from SEQ ID NO:1 to SEQ ID NO:18, wherein a total of no more than three amino acids are substituted, deleted, and/or inserted.

7. The composition of claim 6, wherein said protein comprises an amino acid sequence selected from SEQ ID NO:1 to SEQ ID NO:18.

8. A compound isolated from a Gigartina species, wherein said compound has a molecular weight greater than 3,000 Daltons, and wherein said compound exhibits one or more anti-viral properties.

9. The composition of claim 1, wherein the Gigartina species is selected from the group consisting of Gigartina skottsbergii Setchell & Gardner 1936, Gigartina intermedia, Gigartina exasparata, Gigartina acicularis, Gigartina pistillata, Gigartina radula, Gigartina stellata, and Gigartina acicularis.

10. The compound of claim 8, wherein said compound inhibits viral growth.

11. The compound of claim 8, wherein said compound inhibits influenza replication.

12. The compound of claim 8, wherein said compound comprises a protein.

13. The compound of claim 12, wherein said protein comprises:
1) an amino acid sequence selected from SEQ ID NO:1 to SEQ ID NO:18; or
2) an amino acid sequence that has amino acid substitution of, deletion from, and/or insertion into a sequence selected from SEQ ID NO:1 to SEQ ID NO:18, wherein a total of no more than three amino acids are substituted, deleted, and/or inserted.

14. The compound of claim 13, wherein said protein comprises an amino acid sequence selected from SEQ ID SEQ ID NO:1 to SEQ ID NO:18.

15. A method for treating or preventing a viral infection in a mammalian subject, comprising administering to the subject in need thereof:
a) a composition comprising an extract of a Gigartina species, wherein said extract comprises a component having a molecular weight greater than 3,000 Daltons, and wherein said extract exhibits one or more anti-viral properties; or
b) a compound isolated from a Gigartina species, wherein said compound has a molecular weight greater than 3,000 Daltons, and wherein said compound exhibits one or more anti-viral properties.

16. The method of claim 15, wherein the Gigartina species is selected from the group consisting of Gigartina skottsbergii Setchell & Gardner 1936, Gigartina intermedia, Gigartina exasparata, Gigartina acicularis, Gigartina pistillata, Gigartina radula, Gigartina stellata, and Gigartina acicularis.

17. The method of claim 15, wherein said compound comprises a protein.

18. The method of claim 17, wherein said protein comprises:
1) an amino acid sequence selected from SEQ ID NO:1 to SEQ ID NO:18; or
2) an amino acid sequence that has amino acid substitution of, deletion from, and/or insertion into a sequence selected from SEQ ID NO:1 to SEQ ID NO:18, wherein a total of no more than three amino acids are substituted, deleted, and/or inserted.

19. The method of claim 18, wherein said protein comprises an amino acid sequence selected from SEQ ID SEQ ID NO:1 to SEQ ID NO:18.

20. The method of claim 15, wherein the viral infection is influenza.

21. A method for obtaining an extract having antiviral activity against one or more viruses of interest, comprising:
a) providing one or more biological extracts;
b) carrying out a primary screening on each extract, comprising:
i) evaluating cytopathic effects (CPE) of a virus on host cells in the presence of each extract in vitro relative to a control;
ii) carrying out a quantitative cell viability assay and, optionally, crystal violet staining;
c) carrying out a secondary screening on each extract identified as exhibiting protection against CPE in the primary screening, comprising
i) carrying out a dose response assay on each extract;
ii) carrying out a plaque reduction assay on each extract; and
iii) carrying out one-step virus progeny production;
iv) carrying out a selectivity evaluation comprising a cytotoxicity assay.

22. The method of claim 21, further comprising repeating (b) and (c) one or more times on each extract determined to have antiviral activity, using a different virus of interest.

23. The method of claim 21, further comprising repeating one or more times the primary screening of (b) on each extract identified as exhibiting protection against CPE in the primary screening.

24. The method of claim 21, wherein the extract is identified as exhibiting protection against CPE in the primary screening if the extract provides at least 50% protection at 100 μg/mL.

25. The method of claim 21, wherein the primary screening of (a), the secondary screening of (b), or both (a) and (b) carried out on a plurality of serial dilutions of the extract.

26. The method of claim 21, wherein the extract is a bacterial extract.

27. The method of claim 21, wherein the extract is an algae extract.

28. The method of claim 21, wherein the extract is a marine extract.

29. The method of claim 21, wherein the virus of interest is influenza virus.

30. The method of claim 21, wherein the virus of interest is selected from influenza A H1N1, H3N2, H5N1, or H7N1 virus.


SEQUENCE LISTING (18 sequences)

SEQ ID NO: 1 - protein, 14 aa, Gigartina skottsbergii
TLEVEASDTIENVK

SEQ ID NO: 2 - protein, 13 aa, Gigartina skottsbergii
QDKEGIPPDQQRL

SEQ ID NO: 3 - protein, 28 aa, Gigartina skottsbergii
QLEDGRTLSDYNIQKESTLHLVLRLRGG

SEQ ID NO: 4 - protein, 10 aa, Gigartina skottsbergii
NGEFIEITEK

SEQ ID NO: 5 - protein, 28 aa, Gigartina skottsbergii
LGVDVYAVSTDTHFTHKAWHSSSETIAK

SEQ ID NO: 6 - protein, 13 aa, Gigartina skottsbergii
YAMIGDPTGALTR

SEQ ID NO: 7 - protein, 23 aa, Gigartina skottsbergii
ATFVVDPQGIIQAIEVTAEGIGR

SEQ ID NO: 8 - protein, 13 aa, Gigartina skottsbergii
AAYVGGSDLQALK

SEQ ID NO: 9 - protein, 7 aa, Gigartina skottsbergii
DGEIILR

SEQ ID NO: 10 - protein, 14 aa, Gigartina skottsbergii
TVDLDNEQAVAFTK

SEQ ID NO: 11 - protein, 10 aa, Gigartina skottsbergii
FIAYANDLAR

SEQ ID NO: 12 - protein, 19 aa, Gigartina skottsbergii
ASELGYSDVHLLLGNDGLR

SEQ ID NO: 13 - protein, 9 aa, Gigartina skottsbergii
NEEAMNFVK

SEQ ID NO: 14 - protein, 8 aa, Gigartina skottsbergii
YMDFFAGK

SEQ ID NO: 15 - protein, 13 aa, Gigartina skottsbergii
FAEYANTLAAMAK

SEQ ID NO: 16 - protein, 9 aa, Gigartina skottsbergii
TALNYNLNR

SEQ ID NO: 17 - protein, 9 aa, Gigartina skottsbergii
TALNYNLNR

SEQ ID NO: 18 - protein, 18 aa, Gigartina skottsbergii
FGPYGGSGGSANTLSNVK